(19) United States
(12) Patent Application Publication
(10) Pub. No.: US 2025/0152808 A1
(43) Pub. Date: May 15, 2025

(54) DEVICE FOR DELIVERING INSULIN WITH BASEPLATE AND INTEGRATED MEMS MICROPUMP AND PRESSURE SENSOR

(71) Applicant: AITA BIO INC., New York, NY (US)

(72) Inventor: Dilan Casanovas Mack, New York, NY (US)

(21) Appl. No.: 18/840,045

(22) PCT Filed: Mar. 3, 2023
(86) PCT No.: PCT/US2023/014496

§ 371 Date: Aug. 20, 2024

Related U.S. Application Data
(60) Provisional application No. 63/317,507, filed on Mar. 7, 2022.

(51) Int. Cl. A61M 5/142

(52) U.S. Cl. CPC A61M 5/14244; A61M 2202/0007; A61M 2202/04; A61M 2205/0244; A61M 2205/3327; A61M 2205/3331

(57) ABSTRACT

A device for delivering medicament to a user, the device configured to be mounted to the user, the device including a baseplate comprising: a first opening and second opening to align with a first port and second port of a micropump, respectively; a first fluid channel in fluid communication with the second opening of the baseplate; and a platform for receiving a pressure sensor, the platform having an opening that communicates with the first fluid channel to enable the pressure sensor to sense pressure in the fluid channel.

### CROSS REFERENCE TO RELATED APPLICATIONS

This application claims priority to U.S. provisional application No. 63/317,507, filed Mar. 7, 2022 entitled “Device for Delivering Insulin with Baseplate and Integrated MEMS Micropump and Pressure Sensor” which is incorporated by reference herein.

### FIELD OF THE INVENTION

The present invention relates to a device for delivering insulin with a baseplate and an integrated MEMS micropump and pressure sensor.

### BACKGROUND OF THE INVENTION

Insulin pumps help people with diabetes to conveniently manage their blood sugar. These devices deliver insulin at specific times. Insulin patch pumps or pods are one type of insulin pump. The patch pumps are wearable devices that adhere to the skin of a user using an adhesive patch. The patch pumps are controlled wirelessly with a handheld controller. The patch pumps deliver insulin from a chamber and internal cannula based on separately acquired CGM sensor readings. The fluidic components within the patch pumps are typically significant in number to achieve proper fluid delivery. Component integration for such pumps are complex and current solutions are thus large and expensive. This complication risks device malfunction.

### SUMMARY OF THE INVENTION

A device for delivering insulin with a baseplate and an integrated MEMS micropump and pressure sensor is disclosed.

In accordance with an embodiment of the present disclosure, a device for delivering medicament to a user, the device configured to be mounted to the user, the device including a baseplate comprising: a first opening and second opening to align with a first port and second port of a micropump, respectively; a first fluid channel in fluid communication with the second opening of the baseplate; and a platform for receiving a pressure sensor, the platform having an opening that communicates with the first fluid channel to enable the pressure sensor to sense pressure in the fluid channel.

In accordance with yet another embodiment of the disclosure, a device for delivering medicament to a user, the device comprising: a micropump configured to pump medicament into a user, the micropump including an inlet port to receive medicament and an outlet port to release the medicament within the micropump; a baseplate supporting the micropump, the baseplate including a first channel configured as a fluid path for the medicament and configured to fluidly communicate with the outlet port of the micropump and a catheter for delivering the medicament to the user; and a pressure sensor mounted on the baseplate and configured to communicate with the channel to enable pressure sensing of fluid through the channel.

In accordance with yet another embodiment of the disclosure, a device for delivering medicament to a user, the device configured to be mounted to the user, the device including a baseplate comprising: a first opening and second opening to align with a first port and second port of a micropump, respectively; a fluid channel in fluid communication with the second opening and configured to communicate with a catheter for delivering the medicament to the user; and a first side for supporting a pressure sensor and including an opening that communicates with the fluid channel to enable the pressure sensor to sense pressure in the fluid channel.

### BRIEF DESCRIPTION OF DRAWINGS

FIG. 1 depicts a perspective view of an example baseplate of a device for delivering insulin to a user including an integrated micropump.

FIGS. 2-3 depict perspective views of a pressure sensor to be used in the baseplate shown in FIG. 1.

FIG. 4 depicts a top sectional view of the baseplate shown in FIG. 1.

FIG. 5 depicts a sectional perspective view of the baseplate shown in FIG. 1.

FIG. 6 depicts a bottom sectional view of the baseplate shown in FIG. 1.

FIG. 7 depicts a perspective view of the baseplate shown in FIG. 1 along with the integrated micropump and pressure sensor.

FIG. 8 depicts a perspective sectional view of the baseplate shown in FIG. 1 along with the integrated micropump and pressure sensor.

FIG. 9 depicts a block diagram of example components of a device for delivering insulin.

### DETAILED DESCRIPTION OF THE INVENTION

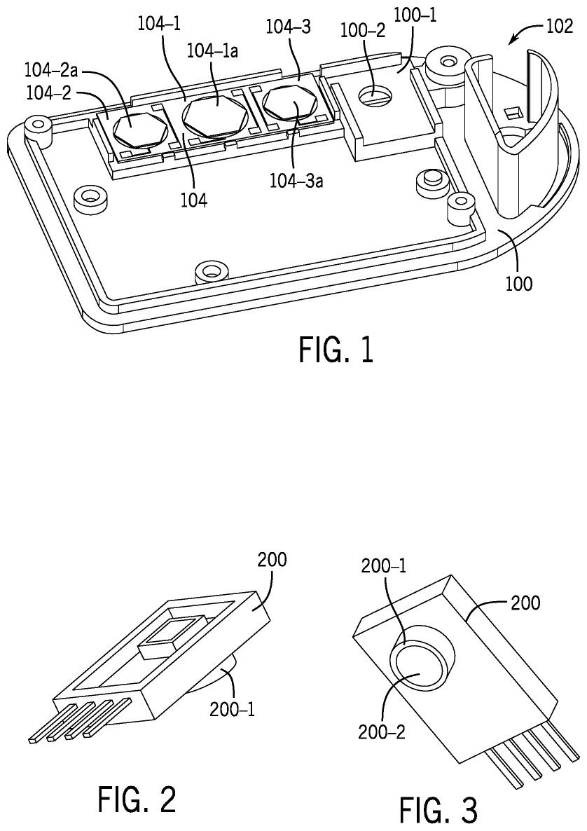

FIG. 1 depicts a perspective view of example baseplate 100 of device 102 (or pod) for delivering insulin to a user. For the purpose of illustrating details of baseplate 100, device 102 depicts only a few components thereof including micropump 104 as described in more detail below.

Device 102 (pod) is configured as a wearable apparatus, that is part of an infusion system for diabetes management, in which continuous glucose monitoring (CGM), insulin delivery and control functionality are provided to ensure insulin is delivered at very precise rates. Device 102 however may be configured to infuse medication/fluids or medicaments to a user other than insulin. Medication or medicament may include small molecule pharmaceutical solutions, large molecule or protein drug solutions, saline solutions, blood or other fluids known to those skilled in the art. Insulin is an example fluid and described below with respect to device 102. However, device 102 may be used in other environments known to those skilled in the art.

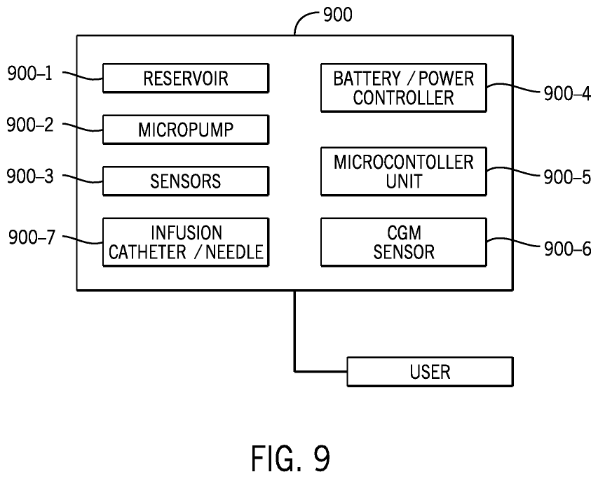

In addition to baseplate 100, device 102 incorporates several components or modules within a housing (not shown) such as micropump 104 as well as a reservoir for storing the insulin, control circuitry (integrated circuit—IC), battery for powering the IC, an insulin needle and a continuous glucose monitoring (CGM) sensor (to name a few). These components are not shown. Example components of a device for delivering medicament such as device 102 are shown in FIG. 9 and described in detail below.)

In this example, micropump 104 is a MEMS (micro-electro-mechanical systems) device, as known to those skilled in the art, that can be used for pumping fluid, valves used for regulating flow, actuators used for moving or controlling the micropump and valves and/or sensors used for sensing pressure and/or flow. The MEMS device incorporates one or more piezoelectric elements or devices (also known herein as piezoelectric transducers), as known to those skilled in the art. Example piezoelectric devices include piezoelectric actuators and various types of MEMS sensors. The piezoelectric devices function as the active element(s) of a pump for pumping fluid and valves for preventing fluid flow and/or a sensor for sensing pressure or flow. (However, various types of MEMS sensors can be used as the sensing elements of the architecture.) Further, other MEMS or non-MEMS structures or technology may also be used to achieve desired results as known to those skilled in the art.) However, the micropump may be constructed of other technologies as known to those skilled in the art.

In some additional detail, in this example, micropump 104 is a cavity substrate that includes a cavity defined by top and bottom wafers (e.g., silicon on insulator and silicon wafers or layers as known to skilled in the art). The top wafer functions as a membrane for the three chambers in this example (or could be any number of chambers). The bottom wafer includes inlet and outlet ports (not shown) that communicate with valve chambers of the cavity via channels that extend through the bottom wafer. Micropump 104 includes a pump 104-1 section and two valve 104-2, 104-3 sections that function together to pump fluid through the three chambers of the cavity.

Pump section 104-1 include piezoelectric actuator (transducer) 104-1a and valve sections 104-2, 104-3 include piezoelectric actuators (transducers) 104-2a, 104-3a respectively that are layered as shown on the top wafer. A metallization and conductive epoxy layer may be used to bind piezoelectric actuators 104-1a, 104-2a, 104-3a to the top wafer as known to those skilled in the art. Piezoelectric actuator 104-1a functions to pump or deform/bend the top wafer (silicon layer) to draw into or displace liquid contents into the pump cavity chamber from either port as desired. Micropump 104 also includes valve seats (not shown) that are configured to extend into valve cavity chambers and define the introduction of channels and the inlet and outlet ports.

Valve sections 104b, 104c are configured as piezoelectric microvalves that function as active valves. Piezoelectric actuators 104-2a, 104-3a are configured to compress against the top wafer (membrane) to reach and seal the valve seats to thereby discontinue flow through the inlet and outlets ports, respectively as needed for proper pump performance, as known to those skilled in the art. (Note that a micropump may include any number of pumps and/or valves as described herein).

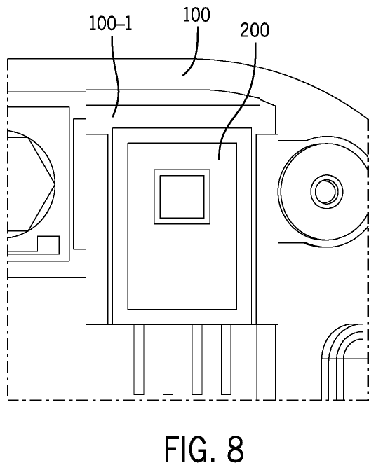

In the example shown in FIGS. 1 and 4-7, baseplate 100 acts as the chassis of device 102 and it is configured to integrate micropump 104 (and pressure sensor 200) onto such chassis as shown. Baseplate 100 includes a platform or sectional part 100-1 to receive pressure sensor 200 (described below.) The fluidic connections are made between micropump 104 and platform 100a of baseplate 100. (Platform 100-1 is also described in more detail below.) Baseplate 100 has the fluidic channels molded directly therein so that when the bonding between micropump 104 and baseplate 100 is complete, there is no other step or structure necessary for the fluidic seal to be leak free between micropump 104 and baseplate 100.

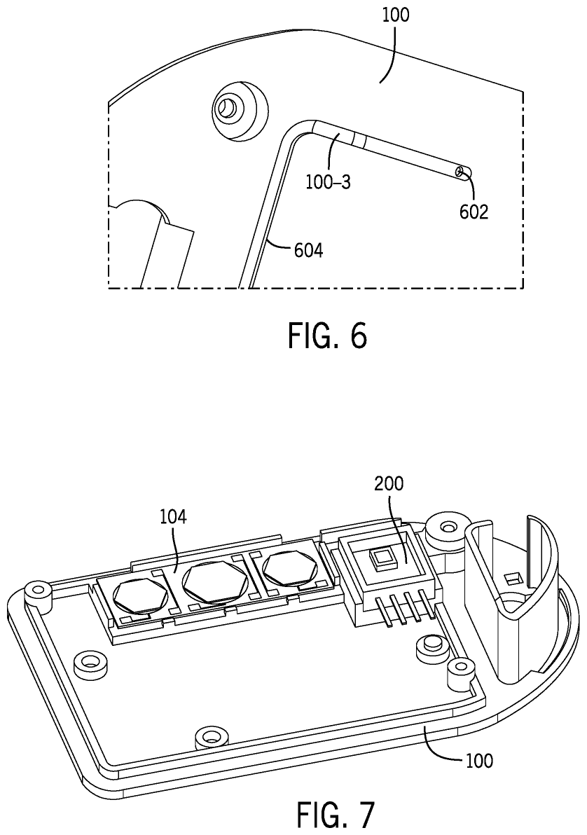

Referring to FIG. 6, the output port of micropump 104 directly communicates with hole 602 in baseplate 100, which ultimately communicates with channel 604 (described in more detail below) on the bottom surface (or side) of baseplate 100. In this way, device 102 as disclosed herein combines MEMS packaging with the fluidic connections necessary to close the fluid path between a reservoir of the device 102 and the needle or cannula that delivers the medication (drug) to the patient. As a result, biocompatible fluidic connections are achieved and baseplate 100 of device 102 (medical device) is used as part of the bottom packaging of a micropump such as micropump 106.

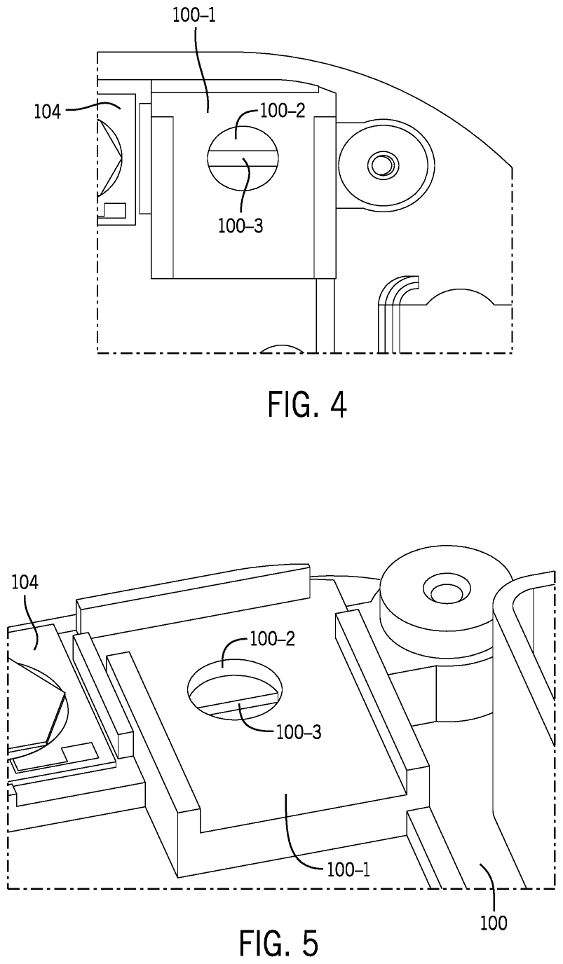

FIGS. 2-3 depict perspective views of a fully packaged pressure sensor 200 to be used in the baseplate 100 shown in FIG. 1. In this example, on the bottom of sensor 200, there is a round protruding tube section 200-1 that is filled with biocompatible gel that protects the silicon membrane of the MEMS pressure sensor die 200-2. When pressure is applied to that gel, pressure senor 200 can detect it. This is an example pressure sensor that can be developed or commercially available. Those skilled in the art know however that other pressure sensors may be employed to achieve desired results.

Baseplate 100 includes platform 100-1 described above. Platform 100-1 incorporates an opening 100-2 to a channel within platform 100-1. Platform 100-1 is configured in shape and dimension to receive and fit pressure sensor 200 with its tube section 200-1 (described below) extending through opening 100-2 and into the channel that extends through platform 100-1. As best seen in FIG. 6, platform 100 has a fluid channel 604 on the bottom thereof that extends from hole 602 that communicates with the output port of micropump 104. This fluid channel 604 is sealed by heat staking a film to it. After sealing this fluidic channel 604, there is an opening or section 100-3 of the channel that comes into contact with pressure sensor 200 to enable sensing functionality. Further structurally, a tube is connected from a reservoir to an opening/channel of baseplate 100 that leads to an inlet (not shown) of micropump 104. Once the drug fluid is pumped out of micropump 104 (MEMS chip), it travels through channel 604 in baseplate 100 (passing pressure sensor 200) that ends up in a tubing that is connected to the needle/cannula of device 102.

In even more detail, section 100-3 is a section of channel 604 that communicates or comes into contact with the silicon membrane of pressure tube 200-1 of sensor 200 to enable it to sense pressure quickly in the fluid path as an occlusion is formed. An O-ring is positioned around pressure sensor 200 that comes into contact with baseplate of device 102. When pressure is applied, the o-ring creates a fluid seal between the pressure sensor 200 and baseplate 100, ensuring the fluidic path is maintained. (Pressure sensor 200 may be held in place with biocompatible adhesive or a cover.) In this way, this integration allows device 102 to monitor pressure changes and behavior quickly in the fluidic channel through the life of device 102. Alternatively, a biocompatible adhesive tape can be used to secure pressure sensor 200 to baseplate 100 in order to fluidically seal the system.

In summary, with this design, sensor 200 is put directly in the fluid pathway within a rigid (non-compliant) component, i.e., baseplate 100 itself, near micropump 102. This ultimately avoids sensing along compliant components such as tubing where occlusion sensing is more difficult (as non-compliant components expand). In addition, the attachment of micropump 102 onto baseplate 100 serves two purposes. First, baseplate 100 acts as a base of the packaging and a protection layer. Second, a full integration of micropump 104 into the fluidic system of the overall medical device is achieved. This ultimately enables pressures sensor 200 to be integrated easily into the fluid path as well.

FIG. 9 depicts a block diagram of example components of device 900 for delivering insulin to a user (device 900 part of an infusion system as described above). (Device 102 above is numbered as device 900 in FIG. 9.) Specifically, device 900 incorporates several components or modules (not shown) in the fluidic pathway including reservoir 900-1 for storing the insulin, micropump 900-2 (as described hereinabove) for pumping the insulin or other medicament, sensors 900-3 (e.g., pressure) for sensing various parameters in the system and a user and tubing connecting infusion catheter or infusion needle 900-7 to reservoir 900-1. Device 900 also includes battery and power controller 900-4 and microcontroller unit (MCU) 900-5.

Device 900 further includes CGM sensor 900-6. CGM or continuous glucose monitoring, as known to those skilled in the art, tracks user glucose levels and permits those levels to be used in algorithms that control flow rate. MCU 900-5 controls the operation of micropump 900-2. Infusion needle 900-7 and CGM sensor 900-6 are shown as separate components in FIG. 4 for illustration purposes. Infusion catheter or needle 900-7 and CGM sensor 900-6 may be integrated or may be separate (individually).

Reservoir 900-1 is configured to receive and store insulin (or other medicament) for its delivery over a course of about three days, or as needed. However, reservoir size may be configured for storing any quantity of fluid as required.

MCU 900-5 electronically communicates with sensors 900-3 and micropump 900-2 as well as the CGM sensor 900-6, as the monitoring components. Among several functions, MCU 900-5 operates to control the operation of micropump 900-2 to deliver insulin through infusion catheter or infusion needle 900-7 from reservoir 900-1 at specific doses, i.e., flow rates over specified time intervals, based on CGM data converted to desired flow rate via control algorithms.

Battery and power controller 900-4 controls the power to MCU 900-5 and micropump 900-2 to enable those components to function properly as known to those skilled in the art. CGM sensor 900-6 is powered by battery and power controller 900-4 through MCU 900-5.

FIG. 9 depict device 900 with only a few components. Those skilled in the art know that device 900 include additional components.

It shall be understood that this disclosure teaches examples of the illustrative embodiments and that many variations of the invention can easily be devised by those skilled in the art after reading this disclosure and that the scope of the present invention is to be determined by the claims below.

What is claimed is:

1. A device for delivering medicament to a user, the device configured to be mounted to the user, the device including a baseplate comprising:
a first opening and second opening to align with a first port and second port of a micropump, respectively;
a first fluid channel in fluid communication with the second opening of the baseplate; and
a platform for receiving a pressure sensor, the platform having an opening that communicates with the first fluid channel to enable the pressure sensor to sense pressure in the fluid channel.

2. The device of claim 1 further comprising a housing configured to engage the baseplate to form an interior therein.

3. The device of claim 1 wherein the baseplate includes a top surface as part of the platform and bottom surface opposing the top surface, and wherein the first fluid channel is formed along a bottom surface of the baseplate.

4. The device of claim 1 wherein the baseplate further comprises a first port in fluid communication with the fluid channel and configured for fluid communication with a catheter.

5. The device of claim 1 further comprising second port and a second fluid channel in fluid communication with the second port the first opening aligned with first port of micropump, the second port in fluid communication with a reservoir.

6. The device of claim 1 wherein the medicament is insulin.

7. A device for delivering medicament to a user, the device comprising:
a micropump configured to pump medicament into a user, the micropump including an inlet port to receive medicament and an outlet port to release the medicament within the micropump;
a baseplate supporting the micropump, the baseplate including a first channel configured as a fluid path for the medicament and configured to fluidly communicate with the outlet port of the micropump and a catheter for delivering the medicament to the user; and
a pressure sensor mounted on the baseplate and configured to communicate with the channel to enable pressure sensing of fluid through the channel.

8. The device of claim 7 further wherein the baseplate includes a platform for receiving the pressure sensor, wherein the platform including an opening that communicates with the channel to enable the pressure sensor for sense pressure within the channel.

9. The device of claim 7 wherein the baseplate includes a top surface as part of the platform and a bottom surface opposing the top surface, and wherein the fluid channel is formed along a bottom surface of the baseplate.

10. The device of claim 7 further comprising the catheter for delivering the medicament to the user.

11. The device of claim 7 wherein the baseplate includes a first port configured to communicate with the first channel and the catheter.

12. The device of claim 11 wherein the baseplate further includes a second port and a second fluid channel in fluid communication with the second port, the second port in fluid communication with a reservoir.

13. The device of claim 7 wherein the micropump is a MEMs device.

14. The device of claim 7 wherein the medicament is insulin.

15. A device for delivering medicament to a user, the device configured to be mounted to the user, the device including a baseplate comprising:
a first opening and second opening to align with a first port and second port of a micropump, respectively;
a fluid channel in fluid communication with the second opening and configured to communicate with a catheter for delivering the medicament to the user; and
a first side for supporting a pressure sensor and including an opening that communicates with the fluid channel to enable the pressure sensor to sense pressure in the fluid channel.

16. The device of claim 15 wherein the baseplate further comprises a platform for receiving the pressure sensor.

17. The device of claim 15 wherein the first side includes a port that communicates with the fluid channel and configured to communicate with the catheter.

18. The device of claim 15 wherein the baseplate further comprises a second side that includes the fluid channel.
